(19) United States
(12) Patent Application Publication
(10) Pub. No.: US 2019/0314192 A1
(43) Pub. Date: Oct. 17, 2019

(54) Closed Loop Respiratory Monitoring System For Sleep Quality Characterization

(71) Applicant: MC10, Inc., Lexington, MA (US)

(72) Inventors: Milan Raj, Natick, MA (US); Ryan McGinnis, Winooski, VT (US)

(21) Appl. No.: 16/345,907

(22) PCT Filed: Oct. 31, 2017
(86) PCT No.: PCT/US17/59210

§ 371 Date: Apr. 29, 2019

Related U.S. Application Data
(60) Provisional application No. 62/415,255, filed on Oct. 31, 2016.

(51) Int. Cl. A61F 5/56; A61B 5/0205; A61B 5/00

(52) U.S. Cl. CPC A61F 5/56; A61B 5/0205; A61B 5/746; A61B 5/0816; A61B 5/0002; A61B 2562/0219; A61B 5/4836

(57) ABSTRACT

A system and method for sensing sleep abnormality in a user is disclosed. The system can include an accelerometer sensor in contact with the skin of the user to measure the deflection of the body during respiration and generate a respiration waveform. A controller receives the respiration waveform from the accelerometer sensor to determine a sleep abnormality measurement as a function of the respiration waveform.

### PRIORITY

The present application claims priority to U.S. Provisional Application No. 62/415,255, filed on Oct. 31, 2016. The entirety of that application is hereby incorporated by reference.

### TECHNICAL FIELD

The present disclosure relates generally to respiratory monitoring. More particularly, aspects of this disclosure relate to using sensors attached to a body to measure indicators of the onset of sleep disorders.

### BACKGROUND

Integrated circuits (ICs) are the cornerstone of the information age and the foundation of today's information technology industries. The integrated circuit, a.k.a. “chip” or “microchip,” is a set of interconnected electronic components, such as transistors, capacitors, and resistors, which are etched or imprinted onto a semiconducting material, such as silicon or germanium. Integrated circuits take on various forms including, as some non-limiting examples, microprocessors, amplifiers, Flash memories, application specific integrated circuits (ASICs), static random access memories (SRAMs), digital signal processors (DSPs), dynamic random access memories (DRAMs), erasable programmable read only memories (EPROMs), and programmable logic. Integrated circuits are used in innumerable products, including computers (e.g., personal, laptop and tablet computers), smartphones, flat-screen televisions, medical instruments, telecommunication and networking equipment, airplanes, watercraft and automobiles.

Advances in integrated circuit technology and microchip manufacturing have led to a steady decrease in chip size and an increase in circuit density and circuit performance. The scale of semiconductor integration has advanced to the point where a single semiconductor chip can hold tens of millions to over a billion devices in a space smaller than a U.S. penny. Moreover, the width of each conducting line in a modern microchip can be made as small as a fraction of a nanometer. The operating speed and overall performance of a semiconductor chip (e.g., clock speed and signal net switching speeds) has concomitantly increased with the level of integration. To keep pace with increases in on-chip circuit switching frequency and circuit density, semiconductor packages currently offer higher pin counts, greater power dissipation, more protection, and higher speeds than packages of just a few years ago.

The advances in integrated circuits have led to related advances within other fields. One such field is sensors. Advances in integrated circuits have allowed sensors to become smaller and more efficient, while simultaneously becoming more capable of performing complex operations. Other advances in the field of sensors and circuitry in general have led to wearable circuitry, a.k.a. “wearable devices” or “wearable systems.” Within the medical field, as an example, wearable devices have given rise to new methods of acquiring, analyzing, and diagnosing medical issues with patients, by having the patient wear a sensor that monitors specific characteristics. Related to the medical field, other wearable devices have been created within the sports and recreational fields for the purpose of monitoring physical activity and fitness. For example, a user may don a wearable device, such as a wearable running coach, to measure the distance traveled during an activity (e.g., running, walking, etc.), and measure the kinematics of the user's motion during the activity.

### SUMMARY

One example of a body function that is desirable to monitor is respiration during sleep to detect abnormalities such as sleep apnea. Sleep apnea is a condition that manifests itself by lack of breathing during sleep. Sleep apnea is caused by an obstruction in the airway pipe or by the inability of the body to relay the proper neural signals to induce the respiratory system. Sleep apnea inhibits a person's ability to adequately inspire oxygen while sleeping, causing the heart to work harder to oxygenate the body's vital organs. This condition may be caused by the most common form of sleep apnea, known as Obstructive Sleep Apnea (OSA). One symptom of this condition includes lack of breathing for long seconds or even minutes, caused by a narrowing of the airway that restricts the flow of air into the lungs.

If the duration and frequency of breathing absence were measurable by a sensor and coupled with ECG data from another sensor, inferences regarding the loading on the cardiovascular system may be drawn. Furthermore, if a motion-based sensor were coupled with this measurement to indicate posture and position during sleep, a determination may be made regarding whether the sleep apnea is caused by positional (PP) or non-positional (NPP) OSA. Unfortunately, currently there is no convenient mechanism to continuously monitor a person during sleep for measurements to indicate the occurrence of sleep apnea.

Thus, there is a need for an accurate system to characterize the occurrence of sleep apnea. There is yet another need for a system to provide non-invasive monitoring for the occurrence of sleep apnea. There is another need for a system that allows the detection of sleep apnea for activating an external correction mechanism.

One disclosed example is a sensor system for sensing a sleep abnormality in a user. The system includes an accelerometer sensor in contact with the skin of the user to measure a deflection of the body during respiration and generate a respiration waveform. A controller receives the respiration waveform from the accelerometer sensor to determine a sleep abnormality measurement as a function of the respiration waveform.

Another disclosed example is a method of detecting a sleep abnormality in a user. An accelerometer sensor is attached in contact with the skin of the user to measure a deflection of the body during respiration. A respiration waveform is generated from the accelerometer sensor. A respiration waveform from the accelerometer sensor is received by a controller. A sleep abnormality measurement is determined as a function of the respiration waveform by the controller.

The above summary is not intended to represent each embodiment or every aspect of the present disclosure. Rather, the foregoing summary merely provides an exemplification of some of the novel aspects and features set forth herein. The above features and advantages, and other features and advantages of the present disclosure, will be readily apparent from the following detailed description of representative embodiments and modes for carrying out the present invention when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 shows a system for a system of wearable sensors for detecting and characterizing sleep apnea in a user;

FIG. 2 is a block diagram of the wearable sensor device in FIG. 1;

FIG. 3 is a graph showing the sampled accelerometer signal of one of the sensor devices in FIG. 1; and

FIG. 4 is a flow diagram showing the process of measuring and recording data associated with sleep apnea in the system in FIG. 1.

The present disclosure is susceptible to various modifications and alternative forms, and some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. There are shown in the drawings, and will herein be described in detail, representative embodiments with the understanding that the present disclosure is to be considered as an exemplification or illustration of the principles of the present disclosure and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed: the singular includes the plural and vice versa; and the word “including” means “including without limitation.” Moreover, words of approximation, such as “about,” “almost,” “substantially,” “approximately,” and the like, can be used herein in the sense of “at, near, or nearly at,” or “within 3-5% of,” or “within acceptable manufacturing tolerances,” or any logical combination thereof, for example.

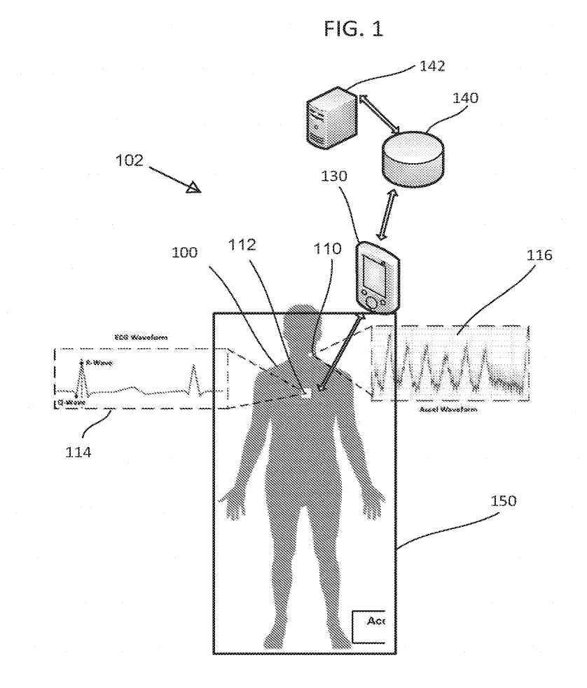

FIG. 1 shows a system 102 that can be worn by a user 100 and can be used for respiratory monitoring and sleep quality characterization. The system 102 can include a wearable sensor device 110 that includes an accelerometer that can detect the motion and vibrations of the user 100 during respiration and an optional wearable sensor device 112 that functions as a heart beat sensor that can, for example, obtain an electro-cardiogram (ECG) signal, a seismocardiogram (SCG) waveform or a PPG signal indicative of the heart beat. The wearable ECG sensor device 112 can be located on the chest of the user 100 in sufficient proximity to measure heartbeat features such as the R-wave of the ECG waveform or the aortic opening feature of the seismocardiogram (SCG) waveform from the heart beat.

The wearable sensor device 110 is preferably positioned on the neck in close proximity to the carotid artery above the left clavicle. However, the sensor device 110 can be located in any area in proximity to the carotid artery such that it can sense the motion or vibration produced by the respiratory airway during respiration. As shown in FIG. 1, the sensor device 110 produces an output signal 116 that is based on sampling of accelerometer signals that detect motion and vibration indicative of user 100 respiration activity (e.g., expansion and contraction of the respiratory airway and sound and/or vibrations resulting from airflow). The wearable ECG sensor device 112 can produce an output signal 114 (e.g., an ECG or similar signal) that is based on sampling the ECG electrodes.

The wearable sensor device 110 and, optionally, wearable ECG sensor device 112 can be in communication with a smart device or hub such as a user device 130. The user device 130 can be a computing device such as a smart phone, a tablet, a laptop or desktop computer, a personal digital assistant or a network of computers (e.g., a cloud or a cluster). The user device 130 can used to control and/or program the wearable sensor device 110 and, optionally, control and/or program wearable ECG sensor 112. Although the wearable sensor devices 110 and 112, as described herein, are used for non-invasive respiratory and/or heart monitoring, each can have other measurement and sensing functions in relation to the user 100. In accordance with some embodiments, the ECG sensor 112 can include an implanted component (e.g., a pace maker).

The data from the wearable sensor device 110 representative of the respiration activity signals (e.g., waveform 116) and, optionally, the data from wearable ECG sensor device 112 representative of the heartbeat signal (e.g., waveform 114) can be uploaded to a cloud storage server 140 periodically (e.g., in time-stamped blocks) or continuously (e.g., streamed) and analyzed by applications running on one or more cloud application servers 142. The data can be processed in real time or using post-processing techniques. The user can access the data, the analysis applications or the output of the applications by accessing the cloud server 142, such as through a website.

As will be explained below, any of the sensors 110 and 112 may be used to sense and store data to determine sleep disorders. The user device 130 can include software that processes the sensed data in order to determine the occurrence and characterization of sleep apnea. Alternatively, on or more cloud applications executed on the cloud application server 160 can process the data received from the sensors 110 and 112 (e.g., via the user device 130) to the determination of the occurrence and characterization of sleep apnea based on the sensed data.

In accordance with some embodiments of the invention, the system shown in FIG. 1 can be used for characterization and evaluation of user respiration patterns and to determine whether a user exhibits symptoms of sleep apnea. As will be explained below, the motion of the user 100 around the carotid artery measured by the accelerometer on the sensor 110 can be used to detect and measure the breathing patterns of the user 100. Derived data and/or metadata such as respiration rate, pulse rate, and snoring frequency and amplitude can be derived from the accelerometer sensor data collected from the wearable sensor device 110. In addition, during obstructive apnea, the airway narrows causing changes in the airflow and these changes in airflow can be detected by the accelerometer as changes in sound or vibration. These sound signals or sound profiles (e.g., characteristic changes in the sounds produced in the airway as it narrows) can also be derived from the accelerometer data produced by the accelerometer sensor of the wearable sensor device 110. The raw sensor data, the derived data and metadata, the sound signals, and/or the sound profiles can analyzed by one or more applications executed by the smart device 130 or one or more applications executed on the cloud server 142 to determine whether the user exhibits symptoms of sleep apnea. For example, the one or more applications can determine that user 100 has stopped (or diminished) breathing for an extended period of time (e.g., motion associated with respiration has been reduced below a threshold or is not discernible by sensor) and/or that user's 100 airway has narrowed (e.g., as indicated by changes in sound vibration of airflow through a narrowed airway). Further, the one or more applications can track (e.g., count) the number of such events over the course of a predetermined time period (e.g., nighttime or the period during which the user 100 is expected to be sleeping). In accordance with some embodiments, the one or more applications can be configured to sound an alarm (e.g., to the user 100 or a person designated by the user 100) if the user 100 stops breathing or experiences very shallow breathing for a predefined period of time.

In addition, the wearable ECG sensor 112 can measure the ECG waveform and can be used to detect irregular patterns in heart rate activity. These irregular heart beat patterns can be analyzed (with or without the respiration data) by one or more applications executed by the smart device 130 or one or more applications executed on the cloud server 142 to determine cardiovascular health issues. For example, changes in the ECG signal coincident with shallow or lack of breathing associated with sleep apnea can be used to determine an impact of the apneic events on the user's heart. More specifically, changes in the amplitude, for example, of the R wave or Q wave can be detected and used to determine a measure of strain on the user's heart.

This respiration and/or sound data can be analyzed in real time by the smart device 130 or cloud server 142 to determine whether the user 100 is suffering from a sleep apnea event (e.g., shallow breathing or lack of breathing associated with sleep apnea). Once this determination is made, the smart device 130 (or the cloud server 142) can sound an alarm or send a signal to an external device, such as an alarm or motor actuators on a mattress, to change the subject's environment and mitigate the apnea event while minimizing the disruption to the user's sleep. In one example, the user device 130 can be programmed to sound an audio alarm when an abnormal breathing pattern associated with sleep apnea is detected. The alarm can be selected such that it will jostle the user 100 promoting a body position change to promote respiration. The user device 130 can also send a signal to a mattress 150 that includes one or more mechanical actuators (e.g., vibration motors) to induce a change in the sleeping position of the user 100 (e.g., raise the user's head or feet). In this manner, the sleep apnea system in FIG. 1 can characterize and even mitigate the symptoms of sleep apnea in a fully closed-loop system.

Further, the system shown in FIG. 1 can use the data collected to predict a sleep apnea event. The accelerometer sensor in sensing device 110 and/or sensing device 112 can be used to detect the user's position while sleeping and monitor changes in respiration over time and use this information to predict the onset of an apnea event based on preprogrammed thresholds or prior apnea event data (e.g. using machine learning algorithms). For example, where a user is sleeping on his/her back and his/her respiration rate begins to slow and/or the motion associated with respiration becomes shallow and/or the sound or vibration of air flowing through the airway changes (e.g., becomes diminished or changes pitch), the system (either the sensing devices 110 and/or 112, the user device 130 or another device can make a sound or cause the bed that supports the mattress 150 to vibrate to stimulate the user to mitigate the onset of the sleep apnea event in real time. The bed can include a controller or computer (e.g., a processor and associated memory) and can include a wireless interface configure to use wireless communications protocols (e.g., BlueTooth Low-Energy, Wi-Fi, ZigBee, etc) to interface with the sensing devices 110, 112, smart device 130, and/or the cloud 142. In using the wireless communications protocol, the computer in the bed can receive a signal from any of these devices, indicating that a sleep apnea event was detected (or predicted) and requires mitigation. In order to mitigate the event, the computer in the bed can actuate motors and gear systems that can cause the bed to vibrate or change the shape of the bed (e.g., raise the head or the feet of the user 100), thus changing the orientation of the user 100 and disturbing the user enough to cause an opening of the airway, mitigating a potential sleep apnea event. This changes the context of the system from reactionary (i.e. informing the user and/or changing the environment to mitigate a sleep apnea event) to proactive (i.e. predicting apnea events and modifying the environment before the onset of an event).

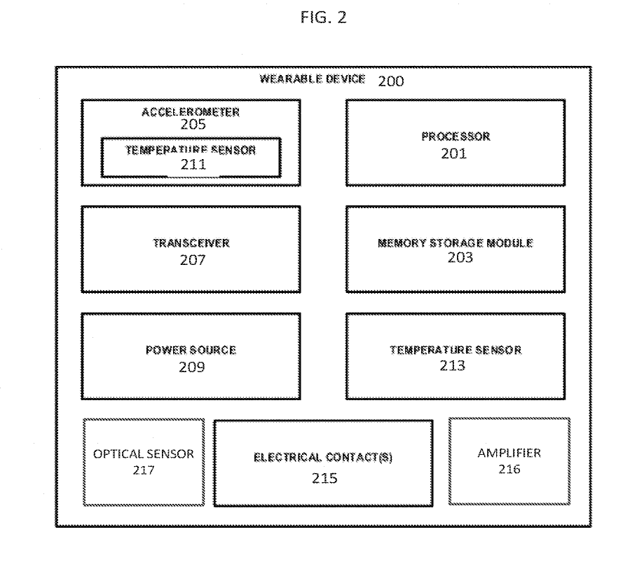

FIG. 2 shows a diagrammatic example of a wearable sensor device 200 such as the sensor device 110 or 112 in FIG. 1 in accord with aspects of the present disclosure. The wearable device 200 can provide conformal sensing capabilities, providing mechanically transparent close contact with a surface (such as the skin or other portion of the body) to provide measurement and/or analysis of physiological information from the user 100. According to some embodiments, the wearable device 200 senses, measures, or otherwise quantifies the motion of at least one body part of a user upon which the wearable device 200 is located. Additionally, or in the alternative, according to some embodiments, the wearable device 200 senses, measures, or otherwise quantifies the temperature of the environment of the wearable device 200, including, for example, the skin and/or body temperature at the location that the wearable device 200 is coupled to the body of a user. Additionally, or in the alternative, according to some embodiments, the wearable device 200 senses, measures, or otherwise quantifies other characteristics and/or parameters of the body (e.g., human or animal body) and/or surface of the body, including, for example, temperature, motion, electrical signals associated with cardiac activity (e.g., ECG), electrical signals associated with muscle activity (e.g., electromyography (EMG)), changes in electrical potential and impedance associated with changes to the skin (e.g., galvanic skin response), electrical signals of the brain (e.g., electroencephalogram (EEG)), bioimpedance monitoring (e.g., body-mass index, stress characterization, and sweat quantification), and optically modulated sensing (e.g., photoplethysmography (PPG) and pulse-wave velocity), and the like.

The wearable device 200 described herein can be formed as a patch. The patch can be flexible and stretchable, and can include stretchable and/or conformal electronics and/or conformal electrodes disposed in or on a flexible and/or stretchable substrate. Alternatively, the wearable device 200 can be rigid but otherwise attachable to a user. In accordance with some embodiments of the invention, the wearable device 200 can include portions that are stretchable and/or conformable and portions that are rigid. Thus, the wearable device 200 can be any device that is wearable on a user, such as coupled to the skin of the user, to provide measurement and/or analysis of physiological information of the user. For example, the wearable device can be adhered to the body by adhesive (e.g. a pressure sensitive adhesive), held in place against the body by tape or straps, or held in place against the body by clothing. The more conformal the sensing device is more likely to stay in position on the skin and produce more reliable and accurate sensor data.

In general, the wearable device 200 of FIG. 2 can include at least one processor 201 connected to one or more associated memory storage modules 203. The wearable device 200 can further include one or more sensors, such as an accelerometer 205 and/or a temperature sensor 213 and/or an optical sensor 217, connected to the processor 201. The wearable device 200 can optionally include one or more wireless transceivers, such as transceiver 207, connected to processor 201 for communicating with other sensor devices such as the sensor devices 110 and 112 or other computing devices such as the user device 130 in FIG. 1. The wearable device 200 can also include a power source 209 connected to the components of the wearable device 200 to power the processor 201, the memory 203 and each of the other components of the wearable device 200. In accordance with some embodiments, the wearable device 200 can be configured to draw power from a wireless connection or an electromagnetic field (e.g., an induction coil, an NFC reader device, microwaves, and light). The wearable device can include, for example, an induction coil and a wireless charging circuit that produces electric power when exposed to an electric or magnetic field to charge the battery and provide power to the wearable device.

The processor 201 can be used as a controller that is configured to control the wearable device 200 and components thereof based on computer program code (e.g., one or more software modules). Thus, the processor 201 can control the wearable device 200 to receive and store sensor data from one or more of the sensors 205, 213, 217. The sensor data can be calibrated and used to determine measures indicative of temperature, motion and/or other physiological data (e.g., ECG, EMG, EEG signals and data), and/or analyze such data indicative of temperature, motion and/or other physiological data according to the principles described herein.

The memory storage module 203 can be configured to save the generated sensor data (e.g., the time when a pulse in blood flow is sensed, accelerometer 205 information, temperature sensor 213 information, or other physiological information, such as ECG, EMG, EEG signals and data) or information representative of acceleration and/or temperature and/or other physiological information derived from the sensor data. Further, according to some embodiments, the memory storage module 203 can be configured to store the computer program code that controls the processor 201. In some implementations, the memory storage module 203 can include volatile and/or non-volatile memory. For example, the memory storage module 203 can include flash memory, static memory, solid state memory, removable memory cards, or any combination thereof. In certain examples, one or more of the memory storage modules 203 can be removable from the wearable device 200. In some implementations, one or more of the memory storage modules 203 can be local to the wearable device 200, while in other examples one or more of the memory storage modules 203 can be remote from the wearable device 200. For example, one or more of the memory storage modules 203 can include the internal memory of a smartphone such as the user device 130 in FIG. 1 that is connected by a wired or wireless connection to the wearable device 200, such as through radio frequency communication protocols including, for example, Wi-Fi, ZigBee, Bluetooth®, medical telemetry and near-field communication (NFC), and/or optically using, for example, infrared or non-infrared LEDs. In such an example, the wearable device 200 can optionally communicate (e.g., wirelessly) with the user device 130 via an application (e.g., program) executing on the user device 130.

In some embodiments, the generated data, including the temperature information, the acceleration information, and/or the other physiological information (e.g., ECG, EMG, EEG etc.), can be stored in one or more of the memory storage modules 203 for processing at a later time. Thus, in some embodiments, the wearable device 200 can include more than one memory storage module 203, such as one volatile and one non-volatile memory storage module 203. In other examples, the memory storage module 203 can store the information indicative of motion (e.g., acceleration information), temperature information, physiological data, or analysis of such information indicative of motion, temperature, physiological data according to the principles described herein, such as storing historical acceleration information, historical temperature information, historical extracted features, and/or historical locations. The memory storage module 203 can also store time and/or date information about when the information was received from the sensor. For example, each data element or block of data elements can be associated with a date and/or time at which it was created.

Although described as the processor 201 being configured according to computer program code in the form of software and firmware, the functionality of the wearable device 200 can be implemented based on hardware, software, or firmware or a combination thereof. For example, the memory storage module 203 can include computer program code in the form of software or firmware that can be retrieved and executed by the processor 201. The processor 201 executes the computer program code that implements the functionality discussed below with respect to determining the on-body status of the wearable device 200, the location of the wearable device 200 on a user, and configuring functionality of the wearable device 200 (e.g., based on the on-body status and sensed location). Alternatively, one or more other components of the wearable device 200 can be hardwired to perform some or all of the functionality.

The power source 209 can be any type of rechargeable (or single use) power source for an electronic device, such as, but not limited to, one or more electrochemical cells or batteries, one or more photovoltaic cells, or a combination thereof. In the case of the photovoltaic cells, the cells can charge one or more electrochemical cells and/or batteries. In accordance with some embodiments, the power source 209 can be a small battery or capacitor that stores enough energy for the device to power up and execute a predefined program sequence before running out of energy, for example, an NFC based sensing device.

As discussed above, the wearable device 200 can include one or more sensors, such as the accelerometer 205, a temperature sensor 213, electrical contacts 215 (e.g., electrical contacts or electrodes), and/or an optical sensor 217. In accordance with some embodiments, one or more of the sensors, such as accelerometer 205, the optical sensor 217 and/or electrical contacts 215, can be separate components from the wearable device 200. That is, the wearable device 200 can be connected (by wire or wirelessly) to each sensor (e.g., accelerometer 205, temperature sensor 213, electrical contacts 215, and optical sensor 217). This enables the wearable device 200 to sense conditions at one or more locations that are remote from the wearable device 200. In accordance with some embodiments, the wearable device 200 can include one or more integral sensors in addition to one or more remote sensors.

The accelerometer 205 measures and/or generates acceleration information indicative of a motion and/or acceleration of the wearable device 200, including information indicative of a user wearing, and/or body parts of the user wearing, the wearable device 200. In accordance with one embodiment, the accelerometer 205 within the wearable device 200 can include a 3-axis accelerometer that generates acceleration information with respect to the x-axis, the y-axis, and the z-axis of the accelerometer based on the acceleration experienced by the wearable device 200. Alternatively, the wearable device 200 can include three independent accelerometers (not shown for illustrative convenience) that each generate acceleration information with respect to a single axis, such as the x-axis, the y-axis, or the z-axis of the wearable device 200. Alternatively, the wearable device 200 can include an inertial measurement unit (IMU) that measures the angular velocity, the orientation, and the acceleration using a combination of one or more accelerometers, gyroscopes, and magnetometers. Thus, although generally referred to herein as an accelerometer 205, the accelerometer 205 can be any motion sensing element or combination of elements that provides acceleration information.

In this example, the accelerometer 205 is an MPU-6500 manufactured by Invensense. According to some embodiments, the accelerometer 205 includes a detection range of ±2 times the force of gravity (Gs). However, the range can vary, such as being ±16 Gs or ±2 Gs. Further, the accelerometer 205 can have a sampling rate of 100 hertz (Hz) such that each second the accelerometer 205 generates 300 points of acceleration information, or 100 points within each axis. However, the sampling rate can vary, such as being 20 Hz to 100 Hz.

According to some embodiments, one or more sensors of the wearable device 200, such as the accelerometer 205, can include a built-in temperature sensor, such as the temperature sensor 211 within the accelerometer 205. For example, the temperature sensor 211 within the accelerometer 205 can be used to calibrate the accelerometer 205 over a wide temperature range and to measure the temperature of the area of the body that the accelerometer 205 is coupled to. Other temperature sensors included with other device components can also be used. Other than the accelerometer 205, and temperature sensor 211, other subcomponents or elements of the wearable device 200 can include one or more microelectromechanical system (MEMS) components within the wearable device 200 that is designed to measure motion or orientation (e.g., angular-rate gyroscope, etc.).

In accordance with some embodiments of the invention, an accelerometer (or an acoustic sensor) such as the accelerometer 205 of the wearable sensor device 200 shown in FIG. 2 can be used to detect and measure a biometric signal known as a seismocardiogram (SCG). The SCG signal can be detected and recorded by the accelerometer 205 of the wearable sensor device 200, for example, due to the tight mechano-acoustic coupling of the wearable sensor device 200 to the skin (or other organ) that enables the device to sense mechano-acoustic waveforms that propagate from the internal organs of the body to the surface of the skin. These waveforms are transduced by the onboard accelerometer 205 of the sensor device 200 into electrical signals that the device can measure, record and store or transmit to other devices such as the user device 130 in FIG. 1. In accordance with some embodiments, the SCG waveform can be more reliable than measurement of the ECG for sensors that are attached at points in the body that are relatively far from the heart or chest of the patient.

Alternatively, or in addition, the wearable device 200 can include a discrete temperature sensor, such as the temperature sensor 213 which can be positioned in a different location from the wearable device 200. The wearable device 200 can use the temperature information detected by the temperature sensor 211 and/or the temperature sensor 213 according to various methods and processes. For purposes of convenience, reference is made below to the temperature sensor 211. However, such reference is not limited to apply only to the temperature sensor 211, but applies to any one or more temperature sensors within or connected to the wearable device 200.

The electrical contacts 215 can be formed of conductive material (e.g., copper, silver, gold, aluminum, a hydrogel, conductive polymer, etc.) and provide an interface between the wearable device 200 and the skin of the user 100, for receiving electrical signals (e.g., ECG, EMG, etc.) from the skin. The electrical contacts 215 can include one or more electrical contacts 215, such as two electrical contacts 215, electrically connecting the skin of the user 100 to an amplifier circuit that can be part of an analog front end circuit 216, to amplify and condition electrical signals (e.g., ECG, EMG, etc.). With two electrical contacts 215, one contact can be electrically configured as a positive contact and the other contact can be electrically configured as a negative contact. However, in some aspects, there may be more than two electrical contacts, such as four electrical contacts 215 (e.g., two positive and two negative electrical contacts), six electrical contacts 215, etc.

The optical sensor 217 can measure the photoplethysmography (PPG) signal when placed on the skin's surface, allowing for the monitoring of various biometrics including, but not limited to, heart rate, respiration, and blood oxygen measurements. The optical sensor 217 can include one or more light emitters that can emit red, green, infrared light or a combination thereof and one or more optical transducers (e.g., photodiode, CCD sensors). Using the one or more optical transducers, the optical sensor 217 can sense the wavelength of the reflected light. In this example, the optical sensor 217 illuminates the skin and the reflected light changes intensity based on the concentration of oxygen in a blood vessel such as an artery or a capillary bed. Thus, a pulse can be detected as a change in the amount of the reflected light due to a change in the concentration of oxygen in a blood vessel and thus the reflected light detected by the optical sensor 217. The system can contain an array of optical sensors in a one-dimensional or two-dimensional grid. In this configuration, the optical sensors can measure reflected light (pulse oxygenation and pulse waveforms) at multiple locations along the vasculature, enabling measurement of time of flight and pulse wave velocity over a given distance (e.g., the separation distance between individual optical sensors. Of course other sensors can be included on the wearable device 200 to detect the pulse such as the accelerometer 205, a pressure sensor, a strain gauge sensor or an acoustic sensor to measure the mechano-acoustic signatures of the pulse.

In addition to the above-described components, the wearable device 200 can include one or more additional components without departing from the spirit and scope of the present disclosure. Such components can include a display (e.g., one or more light-emitting diodes (LEDs), liquid crystal display (LCD), organic light-emitting diode (OLED)), a speaker, a microphone, a vibration motor, a barometer, a light sensor, a photoelectric sensor, or any other sensor for sensing, measuring, or otherwise quantifying parameters and/or characteristics of the body. In other embodiments of the invention, the wearable device 200 can include components for performing one or more additional sensor modalities, such as, but not limited to, hydration level measurements, conductance measurements, and/or pressure measurements. For example, the wearable device 200 can be configured to, or include one or more components that, perform any combination of these different types of sensor measurements, in addition to the accelerometer 205 and temperature sensor 211.

Referring back to the temperature sensor 211, according to some embodiments, the primary purpose of the temperature sensor 211 is for calibrating the accelerometer 205. Accordingly, the temperature sensor 211 does not rely on direct contact to an object to detect the temperature. By way of example, the temperature sensor 211 does not require direct contact to the skin of a user when coupled to the user to determine the skin temperature. For example, the skin temperature affects the temperature information generated by the wearable device 200 without direct contact between the temperature sensor 211 and the skin. Accordingly, the temperature sensor 211 can be fully encapsulated and, therefore, be waterproof for greater durability. The thermal conductivity of the encapsulating material can be selected to control the ability of the temperature sensor 211 to detect the temperature without direct contact.

The wearable device 200 can be constructed of a flexible and/or stretchable printed circuit (e.g., a flex printed circuit board) that can be encapsulated in an elastomer (e.g., silicone, poly urethane, PDMS) that enables the device to stretch and bend. In accordance with some embodiments of the invention, the wearable device 200 can be constructed to have modulus of elasticity (e.g., Young's modulus) similar to the skin of the user or subject. This construction enables the wearable device 200 to be tightly adhered to the skin using a pressure sensitive adhesive such that the sensors in the wearable device are able to detect the slightest motion of the skin as well as the muscles and organ under the skin in the area of the body where the wearable device 200 is attached. This tight coupling can be accomplished using a thin layer (e.g., less than 150 um) of pressure sensitive adhesive and a thin layer (e.g., less than 150 um) of encapsulating material (e.g., silicone). The adhesive and encapsulating materials can be selected to faithfully transmit to the sensors any vibrations or motions from the skin to which it is attached. In accordance with some embodiments of the invention, the sensors in wearable device 200 placed on the neck adjacent the carotid artery can detect the motion of the esophagus expanding and contracting during breathing as well as the vibrations caused by air flowing through the esophagus. In accordance with some embodiments of the invention, the sensors in wearable device 200 placed on the neck adjacent the carotid artery can detect the motion of the user's neck and chest during respiration. In accordance with some embodiments of the invention, the sensors in wearable device 200 placed on the neck adjacent the carotid artery can detect the motion and vibration of the carotid artery as the blood flows through it to detect the heart beat and the heart rate.

The form factor of the wearable device 200 allows positioning and repositioning of the sensor devices at different locations on the body of the user 100 in order to achieve the highest quality of data. In this example, the sensor device 120 placed on the chest of the user 100 in FIG. 1 can be configured in electrocardiogram (ECG) mode in order to receive the ECG signal from the user's heart. The ECG signal can be processed by wearable sensor to detect the R-wave portion of the ECG signal and determine a pulse rate from the time-period measured or calculated between the R-waves (e.g., the peaks of the R-wave). The sensor device 110 captures the motion of the body near the left clavicle using an accelerometer such as the accelerometer 205. Sensor device 110 can be a wearable sensor device 200 with the electrical contacts removed (or disabled) that is coupled to the skin (e.g., by an adhesive) and conforms to the body without applying pressure on the arterial wall that would alter the natural motion or flow (and impede the accuracy of the measured motion and vibration signals). Sensor device 112 can be a wearable sensor device 200 with the electrical contacts that is coupled to the skin (e.g., by an adhesive) and conforms to the body without applying pressure on the arterial wall that would alter the natural motion or flow (and impede the accuracy of the measured ECG, EMG, motion and vibration signals). This tight coupling also reduces the motion artifacts while enabling high resolution and accurate sensing.

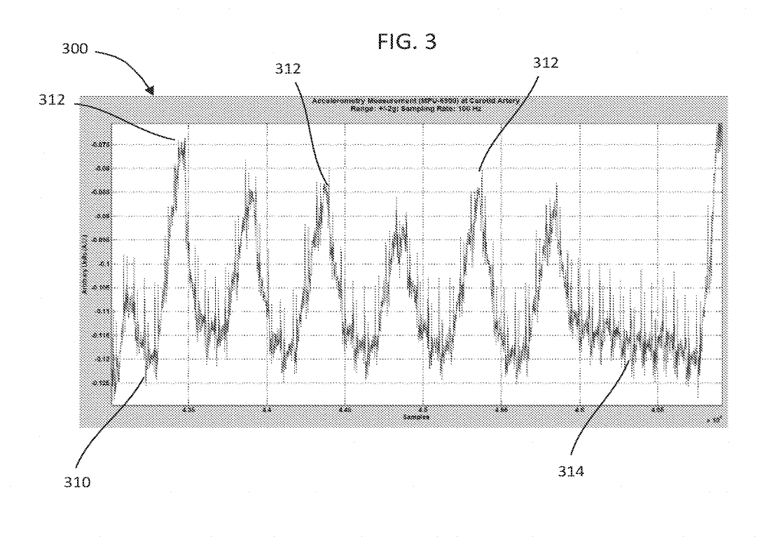

The sleep apnea detection system in FIG. 1 includes the sensor device 110 that measures the motion and vibration of the body around the carotid artery. This motion and vibration directly correlates to the respiration and heart beat patterns of the subject and is used to determine data representative of respiration rate and amplitude, pulse rate, and snoring frequency and amplitude. FIG. 3 is a graph 300 showing a visualization of the accelerometer data collected by the sensor device 110 attached on the clavicle of the user 100 in FIG. 1. A trace 310 is the output of the accelerometer of the sensor device 110, which is a waveform captured during sleep of the user 100. In this example, the accelerometer of the sensor device 112 is sampled at 100 Hz and digitized to 8 bits. A series of peaks 312 signifies normal breathing that occurs at a rate of approximately 0.25 Hz. A period of inactivity 314 without significant peaks indicates an interruption of breathing and likely sleep apnea. In this example, the subject's breathing was arrested for approximately 8 seconds.

In accordance with some embodiments of the invention, the user 100 can attach the sensor 110 on the neck near the left clavicle and the sensor 112 near the heart and left clavicle respectively before sleeping. The sensors 110 and 112 allow the user device 130 to monitor respiration for events related to sleep apnea throughout a sleep period. The sensor 110 measures the motion of the user 100 and detects breathing inactivity from the waveform such as that shown in FIG. 3. The sensor device 110 logs breathing inactivity as a potential sleep apnea event. The sensor 112 measures the ECG waveform and logs the ECG waveform. In this example, the recording and detection of the acceleration waveform and the ECG are performed by the sensors 110 and 112. Alternatively, the data from the sensors 110 and 112 may be transmitted to the user device 130 for detection and logging of breathing inactivity.

The smart device 130 collects data from the sensors 110 and 112 via a wireless communication channel such as a BlueTooth Low Energy signal. Based on the collected data, the user device 130 makes a determination if the event is due to apnea. For example, motion-based data can be used to detect and record the respiration movements of the user 100. An abrupt cessation of respiration, as identified by the motion data can be used to detect a sleep apnea event. This manifests itself by an absence of the waveform morphology found in the waveform 116 (i.e., the steady undulation of the waveform in the respiration frequency range indicates respiration). In addition to this data stream, data from the sensor 112 can be used to augment the analysis by providing ECG data. This can inform the algorithm that runs on the cloud server 142 or user device 130 whether the user's heart is loaded more heavily by the lack of respiration (i.e., the heart must pump more quickly to feed oxygen to the body, compensating for the lack of oxygen in the system). The user device 130 may provide activation of an external device such as an alarm or the actuators on the mattress 150 or a bed to mitigate the sleep apnea event.

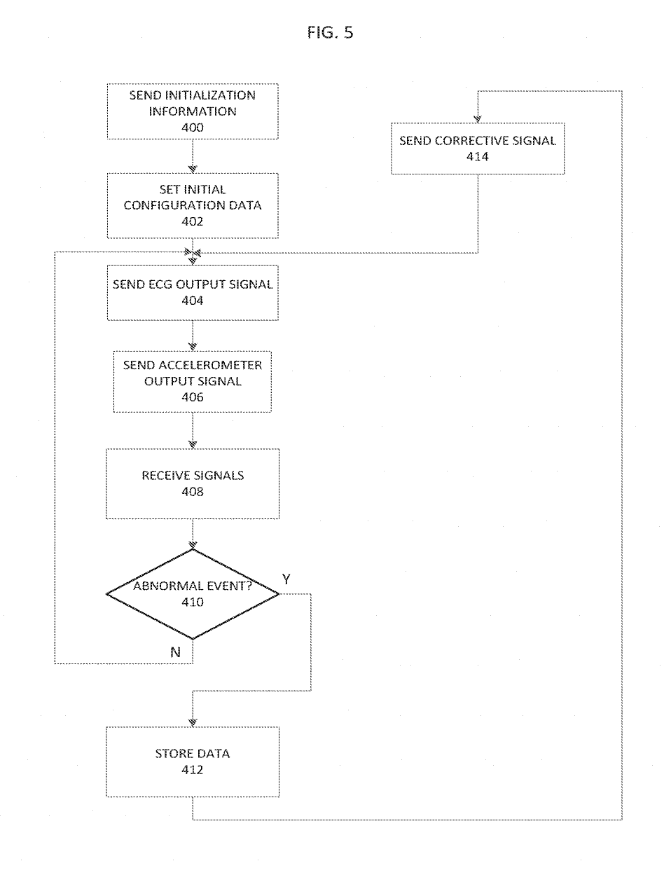

FIG. 4 is a flow diagram of the process of collecting data and determining a sleep apnea event in the system 100 shown in FIG. 1. Handshaking is performed between the user device 130 and the sensor devices 110 and 112 (400). The handshaking involves sending identification information for the sensor devices 110 and 112 and respective MAC addresses to the user device 130. The user device 130 sets initial configuration data such as the location of the sensor devices 110 and 112 on the body, the sampling rate and applicable storage parameters (402).

In this example, the sensor device 112 can continuously (or periodically) send the output of the ECG signal received from the electrical contacts 215 in FIG. 2 to the user device 130 (404). The output of the ECG signal can include one or more samples (e.g., 2, 3, 4, 5, 10, 20, or more samples) associated with a particular timestamp. The sensor device 110 continuously (or periodically) sends an output accelerometer signal to the user device 130 that can include one or more samples (e.g., 2, 3, 4, 5, 10, 20, or more samples) associated with a particular timestamp (406).

The user device 130 receives the ECG output waveform signal from the sensor device 112 and the accelerometer output waveform signal from the sensor device 110 (408). The user device 130 determines whether there is an abnormal event such as an occurrence of an interruption in respiration indicating sleep apnea based on the analysis of the received data (410). If there is no abnormal event, the user device 130 returns to receiving the output signals (408). If an interruption is detected, the user device 130 stores the data from the waveform signals in memory (412). The device 130 then sends a corrective signal to an external device such as an alarm or an actuator (414). The user device 130 then returns to receiving data (408).

The process described in FIG. 4 is a real time determination of the occurrence of sleep apnea by the user device 130. Some or all of the operations described above may be performed by the sensor device 110 or 112. Alternatively, the timestamp data and respective signals may be transmitted to the cloud server 142 and some or all of the above operations may be performed by the cloud server 142. Alternatively, the sensor device 110 or the sensor device 112 may store the waveform data and transmit the stored data periodically to the user device 130 for analysis of sleep interruption or abnormal patterns at a delayed time.

Knowledge of the data relating to sleep abnormalities from the data collected from the sensors 110 and 112 may allow physicians to tailor treatment options for OSA patients. In addition, Central Sleep Apnea (CSA), where the brain fails to relay the appropriate signals to initiate breathing, is a condition that may be differentiated from OSA by data from other sensors on the body. These may include, but are not limited to, motion, ECG, and PPG based sensors. Based on the above background, the system 100 allows accurate and non-invasive characterization of sleep apnea using the sensors 110 and 112, providing a means to potentially predict the onset of sleep apnea and/or augment a user's environment to prevent its persistence throughout the night.

The system 100 may also function as a real time health monitor for at-risk patients and their caregivers. Examples of the former include, but are not limited to: infants at risk for Sudden Infant Death Syndrome (SIDS) and patients with severe forms of sleep apnea, etc. In other embodiments, the system can be used to gauge frequency and efficacy of blood flow through the carotid artery. This can be accomplished by using the data from the accelerometer 205 in FIG. 2 to determine if there is pulsatile blood flowing through the carotid artery. Section 314 of graph 300 shows the spikes corresponding to the blood flow through the artery. In the absence of these spikes, a patient or caregiver can infer that there is a change to the blood flow characteristics of through the carotid artery. This metric has implications for determining a patient's risk for stroke.

In some embodiments, the aforementioned methods include at least those steps enumerated above. It is also within the scope and spirit of the present disclosure to omit steps, include additional steps, and/or modify the order of steps presented herein. It should be further noted that each of the foregoing methods can be representative of a single sequence of related steps; however, it is expected that each of these methods will be practiced in a systematic and repetitive manner.

While particular embodiments and applications of the present disclosure have been illustrated and described, it is to be understood that the present disclosure is not limited to the precise construction and compositions disclosed herein and that various modifications, changes, and variations can be apparent from the foregoing descriptions without departing from the spirit and scope of the invention as defined in the appended claims.

1. A sensor system for sensing a sleep abnormality in a user, the system comprising:
an accelerometer sensor in contact with the skin of the user to measure a deflection of the body during respiration and generate a respiration waveform;
a controller receives the respiration waveform from the accelerometer sensor to determine a sleep abnormality measurement as a function of the respiration waveform.

2. The sensor system of claim 1, further comprising a heart beat sensor in contact with the skin of a user to measure a heart beat waveform, wherein the controller receives the heart beat waveform from the heart beat sensor and determines the sleep abnormality measurement from the heart beat waveform.

3. The sensor system of claim 2, wherein the heart beat sensor is one of an ECG sensor, a SCG sensor or a PPG sensor.

4. The sensor system of claim 1, further comprising an external device in communication with the controller, wherein the controller is operative to control the external device to induce a change in the position of the user.

5. The sensor system of claim 4, wherein the external device is an alarm.

6. The sensor system of claim 4, wherein the external device is a mattress including an actuator to induce a position change of the user.

7. The sensor system of claim 2, further comprising a memory for storing the heart beat waveform and the accelerometer waveform.

8. The sensor system of claim 2, further comprising a transceiver to transmit the heart beat waveform and the accelerometer waveform.

9. The sensor system of claim 8, further comprising a user device in communication with the transceiver to receive the heart beat waveform and the accelerometer waveform, the user device operative to determine the sleep abnormality measurement.

10. The sensor system of claim 1, wherein the accelerometer sensor is on an attachable sensor near the carotid artery.

11. A method of detecting a sleep abnormality in a user comprising:
attaching an accelerometer sensor in contact with the skin of the user to measure a deflection of the body during respiration;
generating a respiration waveform from the accelerometer sensor;
receiving a respiration waveform from the accelerometer sensor by a controller; and
determining a sleep abnormality measurement as a function of the respiration waveform via the controller.

12. The method of claim 11, further comprising:
attaching a heart beat sensor in contact with the skin of a user to measure a heart beat waveform;
generating a heart beat waveform; and
wherein the controller receives the heart beat waveform from the heart beat sensor and determines the sleep abnormality measurement from the heart beat waveform.

13. The method of claim 12, wherein the heart beat sensor is one of an ECG sensor, a SCG sensor or a PPG sensor.

14. The method of claim 11, further comprising providing an external device in communication with the controller, wherein the controller is operative to control the external device to induce a change in the position of the user.

15. The method of claim 14, wherein the external device is an alarm.

16. The method of claim 14, wherein the external device is a mattress including an actuator to induce a position change of the user.

17. The method of claim 11, further comprising storing the respiration waveform in a memory.

18. The method of claim 11, further comprising transmitting the respiration waveform.

19. The method of claim 18, further comprising:
receiving the transmitted respiration waveform via a user device; and
determining the sleep abnormality measurement via the user device.

20. The method of claim 12, wherein the heart beat sensor is on an attachable sensor near the heart; and the accelerometer sensor is on another attachable sensor near the carotid artery.
